(19) United States
(12) Patent Application Publication
(10) Pub. No.: US 2011/0259335 A1
(43) Pub. Date: Oct. 27, 2011

(54) Headgear Support for Respirator Mask

(75) Inventor: Collin Sullivan, North South Wales (AU)

(73) Assignee: AUSTRALIAN CENTRE FOR ADVANCED MEDICAL TECHNOLOGY, Balmain (AU)

(21) Appl. No.: 13/128,237

(22) PCT Filed: Nov. 6, 2009
(86) PCT No.: PCT/AU2009/001439

§ 371 Date: Jul. 12, 2011

(30) Foreign Application Priority Data
Nov. 7, 2008 (AU) 2008905766

(51) Int. Cl. A61M 16/06; A61M 16/00; A42B 1/24

(52) U.S. Cl. 128/205.25; 2/209.13; 128/206.21; 128/207.18

(57) ABSTRACT

A self supporting head mounted harness for mounting a respirator mask on the face of a user, the harness including a harness body having a material stiffness sufficient to enable the harness to be retained and self supported on the head of a user independent of whether or not the mask is attached to the harness.

### FIELD OF THE INVENTION

This invention relates to supporting assemblies for respirator masks and in particular for supporting respirator masks of the type which supply gases, typically air or oxygen, to the airways (nose or nose and mouth) of humans. Respirator masks are particularly suited to applying continuous positive airway pressure to patients for treatment of conditions such as but not limited to sleep apnoea. More particularly, the invention relates to a headgear locatable on a head of a user and which is self, supporting with or without a mask attached, and once set in position, enables a mask to be attached and detached without or with minimal disturbance to the self supporting headgear and the wearer.

### BACKGROUND OF THE INVENTION

Conventional respirator masks consist of a face contacting part which defines an orifice and which fits over the patient's nose and/or mouth and provides a gas tight seal against the patient's skin. The mask includes a manifold part for the delivery of pressurized gases to the patient's nose and/or mouth via a gas delivery tube connected to the manifold. Typically, the manifold part is made from a rigid material to which an adjustable harness, for retaining the mask on a patient's head, is attached. The geometry of the manifold is fixed. Typically, a respirator mask is placed on the patient's face over the mouth and nose and secured by a head mounted harness. The harness transfers forces through the rigid manifold and onto the face contacting part of the mask. The face contacting part is compressed against the patient's face under the effect of the harness forces causing a gas tight seal to form between the face contacting part and the patient's face.

The face contacting part of a conventional respirator mask can be made from a soft flexible material such as silicone rubber, which distorts in one axis (the Z axis) perpendicular to the plane of the patient's face under the loading provided by the harness. The configuration of the harness and mask results in any forces transmitted to the mask being transmitted in the Z direction onto the face contacting part thereby preventing unwanted distortion of the mask in the X-Y plane.

In one commonly known harness arrangement for a conventional respirator mask, there is provided elastic webbing formed from a pliant and flexible plastics or cloth material This includes a plurality of adjustable straps which conform to the shape of a head once sufficient tension is introduced into the straps. The network of straps or webbing is held in position once a mask is attached to at least two of the straps and the straps tensioned to apply sufficient force to the mask to keep it pressed against the user's face. If the tension forces are relieved in the flexible harness webbing, it is no longer self supporting and falls limp away from the users head at the same time relieving locating forces on the mask. One of the problems that arises from use of respirator masks is the un avoidable disturbance to the patient to set the mask in position. This is particularly a problem with infants and patients that are asleep.

According to the known harnessing assemblies, in order to set a mask, access must be gained to the posterior of the head to set the webbing in position before the final step of attaching the mask to the harness. Usually the head of a patient is lifted or at least moved to allow the nurse or doctor attending the patient to set the harness. Typically a harness will have an arrangement whereby straps will lie at each side of the head avoiding the ears. In addition, there is an overhead strap to provide additional support. Each strap of the harness webbing terminates in a free end which has means such as a Velcro strap which engages a connector on the mask. Once the straps are pulled tight, the mask is secured airtight against the face. The method, of fitting just described requires considerable disruption to the patient's head with a similar disruption occurring if and when the mask is removed and repositioned.

In use, as the face contacting portion of the mask is lowered onto a patient's face some areas of the flexible membrane portion will contact some parts of the patient's face before others. Once in place, the mask is compressed tightly against. the face but some mask designs allow the mask to float to a limited extent if the patient's head moves. In masks having a flexible membrane this provides a gas tight seal between the mask and the patient's face. In this way, such conventional masks attempt to form a gas tight seal in a diverse range of patients having different facial contours, which vary significantly in their X-Y-Z topography, at the position of the mask interface.

Setting a mask to its harness can be an uncomfortable experience and causes unwanted disturbance causing pressure on the patient's face and uncomfortable manipulations until the mask is set. According to conventional methods a mask harness can be placed over the patient's head roughly in the desired position to secure the mask following which the mask is connected to the free ends of the harness straps. This might involve a series of trial and error fittings before the right tension force in the webbing of the harness and the correct compression force on the face are achieved. Masks must be set with an optimal compression to satisfy the dual objectives of patient comfort and the creation of an efficient air seal. Many patients find masks uncomfortable particularly where a higher force is required to achieve a seal as sections of the face contacting part can cause discomfort when pressed locally against a Patient's face at the pressures required to create the gas tight seal. Often, a bulky rigid manifold and attachment strap points of a harness can cause discomfort and annoyance in some patients.

An additional problem arises from the use of flexible webbing type harnesses when a patient wearing a mask turns in bed and contacts an object such as a pillow. Reaction forces from the pillow can tighten the straps, cause them to dislodge or cause the head to move relative to the strap thereby displacing the mask and interrupting the air tight seal which prevents optimum therapy being delivered to the patient. This requires re setting of the mask and usually access to the behind the head harnessing and thus further disturbance to the patient.

Constant disturbances of this type make it difficult for a patient to sleep due to uneven pressures on the manifold and the need to continually monitor and rest the harness and mask fitment. A mask harness which is difficult to fit requires more labour and therefore increased labour time and costs. A mask assembly including a manifold and harness made from many parts is typically more expensive to produce than a mask having fewer parts, due to increased costs for the many different parts, and assembly and inventory costs.

When side straps are pulled back across the cheeks in a direction that passes below the ears and the nasal arch flexible strap is pulled toward the top of the head (Z-axis), the mask is pulled onto the face such that the face contacting element orifice encapsulates the subjects nose or nose and mouth and causes the face contacting element to provide an airtight seal between the mask and patient's skin.

It is essential that a respirator mask be fitted optimally for all circumstance of use including during use of both positive and negative gas pressure therapy. Patients using nasal masks may release gas from their mouths as pressurised gas enters a patient's oral cavity which could cause the patient's cheeks to stretch and balloon out causing the mouth to open. It is essential that the harness used on any particular mask will contribute to the creation of an efficient seal and without compromise to patient comfort.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present invention seeks to address and attempt to alleviate at least some of the deficiencies of the existing mask harnessing arrangements described above.

This present invention provides a supporting harness assembly for respirator masks and in particular for supporting respirator masks of the type which supply gases, typically air or oxygen, to the airways (nose or no and mouth) of humans. The invention further provides an improved headgear locatable on a head of a user and which is self supporting with or without a mask attached and once set enables a mask to be detached without disturbance to the self supporting headgear and therefore to the patient to which the headgear is attached.

In its broadest for the present invention comprises:

a self supporting head mounted harness for receiving and retaining patient care equipment and devices, the harness including a harness body having a material stiffness sufficient to enable the harness to be retained and self supported on the head of a user independent of whether or not the equipment or devices are attached to the harness.

In another broad aspect, the present invention comprises:

a supporting head gear for a respirator mask of the type having a manifold and face-contacting components which conform to different shapes to suit differing facial contours when the mask is compressed against the face, wherein the harness comprises a body having at least two members which each terminate at a free end; the free ends having connection means which receive and retain a part of the mask; wherein the harness body is formed from a material which is capable of exerting at least a slight gripping or clamping force against the head of a wearer, such that when the mask is retained by the harness or removed from the harness the harness is self supporting on the wearer's head.

In a further bread form the present invention comprises:

an overhead attachment member for detachable engagement with a self supporting head mounted harness for mounting a respirator mask on the face of a user, the harness including a harness body having a material stiffness sufficient to enable the harness to be retained and self supported on the head of a user independent of whether or not the mask is attached to the harness; the overhead attachment member including an adjustable arm which enables adjustments in the length of the overhead member to accommodate different patient head sizes.

According to a preferred embodiment the free ends of the harness body have a length adjustable assembly which enables an initial length adjustment to accommodate wearers' heads of different sizes. According to a preferred embodiment, the free ends each include a detachable extension arm which is adjustable relative to the free end of the headgear to enable the length adjustments. Preferably the harness is manufactured from a material which allows at least of part of the headgear to undergo elastic displacement to apply a clamping reaction force to the head of a user to hold the harness body in place on the head whether or not there is a mask attached to it.

The extension arms are set relative to a patient's had size to enable a sufficient compression force to he applied to a mask attached to the harness to maintain the airtight seal. The setting will differ from patient to patient. The nature of the connections between the harness extension arms and the mask are such that loadings applied to the mask via the arms are distributed evenly so that distortions of the mask under load will not compromise the required air seal between the user's race and the face contacting part of the mask.

According to one embodiment, the harness includes two free end connection points to the mask. According to an alternative embodiment, the harness includes a third overhead member which terminates in a free end connection assembly which connects directly or indirectly to a pipe on a mask manifold. Preferably, the overhead member includes an adjustable arm which enables adjustments in the length of the overhead member to accommodate different patient head sizes.

Preferably, the third overhead member engages a web or nasal arch strap which is integral to a portion of the gas delivery pipe. Preferably, the manifold is sufficiently flexible to collapse towards the patient's nose when a moderate external force is applied to it.

Preferably, the harness is sufficiently flexible to apply at least a mild clamping force to a wearer's head and sufficiently rigid to enable the harness to sit about the wearer's head whether or not a mask is attached. In order to receive and retain the mask to the free end members of the harness, the mask preferably includes straps or webs which have eyelets or slots which engage the connection assemblies of the free ends of the harness members. Preferably the webs are integral with the mask and join to the side walls of the mask. The slots or eyelets are preferably located at free ends of the webs. Webs extend away from the side wall of the mask, generally with at least one strap on each side of the mask and one strap running along the nasal arch of the forehead.

In use, the mask is connected to the headgear by the application of a tensile force to webs on the mask and engaging the slots to tabs on the free end connecting assemblies of the headgear. The required degree of mask compression on the face of a wearer is pre set by setting the headgear to accommodate a particular head size such that when the webs of the mask engage the connection assemblies on the free ends of the headgear the correct compression force for the mask is achieved satisfying patient comfort and the maintenance an airtight seal between the mask and the patient's face.

Preferably the members which form the harness are manufactured in a mould from suitably stiff but suitably flexible materials to enable achievement of the objections of the invention.

The mask is also preferably sufficiently flexible to enable a carer to remove it or fix it in position without having to adjust any harness connection points where a harness connects to the straps of the mask.

It is preferred that a mask manifold is flexible enough to collapse toward the patients nose when a moderate external force is applied to it With the mask in situ, the manifold can be distorted onto the patient's nose without breaking the airtight seal between the face contacting element and the patients skin. This also means that when a patient turns in sleep and their mask contacts a pillow or some other object the manifold will deform and/or displace rather than be pushed against the patient's face. The harness which is relatively stiff will during patient sleep retain its position on the head such that should be mask require adjustment or removal and/or reapplication this case be done with minimum disruption to the patient.

In a preferred embodiment, mask shaped forming elements may provide multiple attachment points to the harness which can be adjusted in length so as to change the distribution of forces to various areas of the flexible mask, through tension exerted in any specific direction.

In a yet further embodiment, the harness may be varied in its relative stiffness at different points around its attachment position to the perimeter of the head of a wearer so as to apply clamping or gripping forces to enable the harness to be self supporting.

The flexible face contacting element is preferably sufficiently flexible that it substantially collapses onto the patient's face under the normal forces exerted on it by the harness when in situ. The mask will preferably inflate under normal operating pressures when in situ on a patient's face, but will preferably not significantly distend in comparison with the mask's “resting” shape.

The side members of the harness are curved to follow the general shape of the contours of a patient's head and cheeks and to skirt around the ears. The headgear members may be sufficiently rigid to prevent inflation of the patient's cheeks during delivery of positive airway pressure therapy.

According to one embodiment, the device attached to the harness is an air pipe. According to an alternative embodiment, the device attached to the harness is a monitor for measuring a patient health parameter. According to an alternative embodiment, the device attached to the harness is a sensor for determining and recording a patient health parameter. According to an alternative embodiment, the device attached to the harness is a nasal prong or nasal pillow. According to an alternative embodiment, the device is a tube delivering a gas such as oxygen to the patient.

In another broad form the present invention comprises:

a harness assembly for a respirator mask for supplying gas under pressure to an airway of a human:

the mask including a flexible manifold shell, being made of a flexible material, the manifold including means for connection to a gas delivery pipe,

at least two side walls which are at least partially comprised of portions of the manifold shell;

a first mask shape forming element for distributing distortional forces to a substantial portion of one side wall that attaches to or is integral with a significant portion of that one side wall of the mask; and

a second mask shape forming element for distributing distortional forces to a substantial portion of an other side wall that attaches to or is integral with a significant portion of that other side wall of the mask, each mask shape forming element being connected to, or being connectable to, a strap;

a flexible face contacting element defining an orifice to accommodate the nose of the human;

characterised in that the harness assembly comprises:

a body having at least two members which each terminate at a free end; the free ends having connection means which receive and retain a part of the mask; wherein the harness body is formed from a material which is capable of, exerting at least a slight gripping or clamping force against the head of a wearer such that when the mask is retained by the harness or removed from the harness the harness is self supporting on the wearer's head.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of, the invention. In the accompanying illustrations, like reference characters designate the same or similar parts throughout the several views. The following detailed description is, therefore, not to be taken in a limiting sense, and the scope of the present invention is best defined by the appended claims. For instance although the invention is described in the context of an attached respirator mask it will be appreciated that other devices can be attached to the head harness such as but not limited to an air pipe, a monitor for measuring a patient health parameter, a sensor for determining and recording a patient health parameter, a nasal prong or nasal pillow or a tube delivering a gas such as oxygen to the patient.

### BRIEF DESCRIPTION OF THE DRAWINGS

A specific embodiment of the present invention will now be described, by way of example only, and with reference to the accompanying drawings, in which:

FIG. 1 shows a perspective view of a mask mounting harness according to one embodiment;

FIG. 2 shows a top view of the harness of FIG. 1;

FIG. 3 shows an enlarged view of the connection between the headgear and extension arm.

FIG. 4 shows a side elevation of an overhead connecting arm remote form the headgear;

FIG. 4b shows a top view of the overhead connecting arm 30.

FIG. 5 shows a front perspective view of the overhead connecting arm of FIG. 4;

FIG. 6 shows a rear, perspective view of the connecting arm of FIG. 5.

FIG. 7 shows a front perspective view of the harness of FIG. 1 with the overhead connecting arm of FIGS. 4-6 connected thereto.

FIG. 8 shows a top view of the harness of FIG. 7;

FIG. 9 shows a side view of the harness of FIG. 7;

FIG. 10 shows the harness of FIG. 7 mounted on the head of a wearer.

### DETAILED DESCRIPTION

The examples referred to herein are illustrative and are not to be regarded as limiting the scope of the invention. While various embodiments of the invention have been described herein, it will be appreciated that these are capable of modification, and therefore the disclosures herein are not to be construed as limiting of the precise details set forth, but to avail such changes and alterations as fall within the purview of the description.

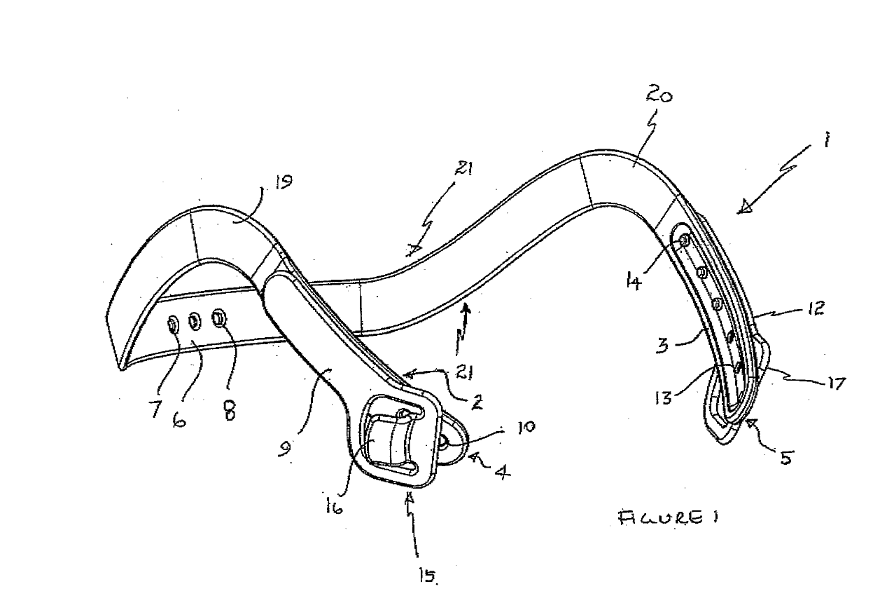

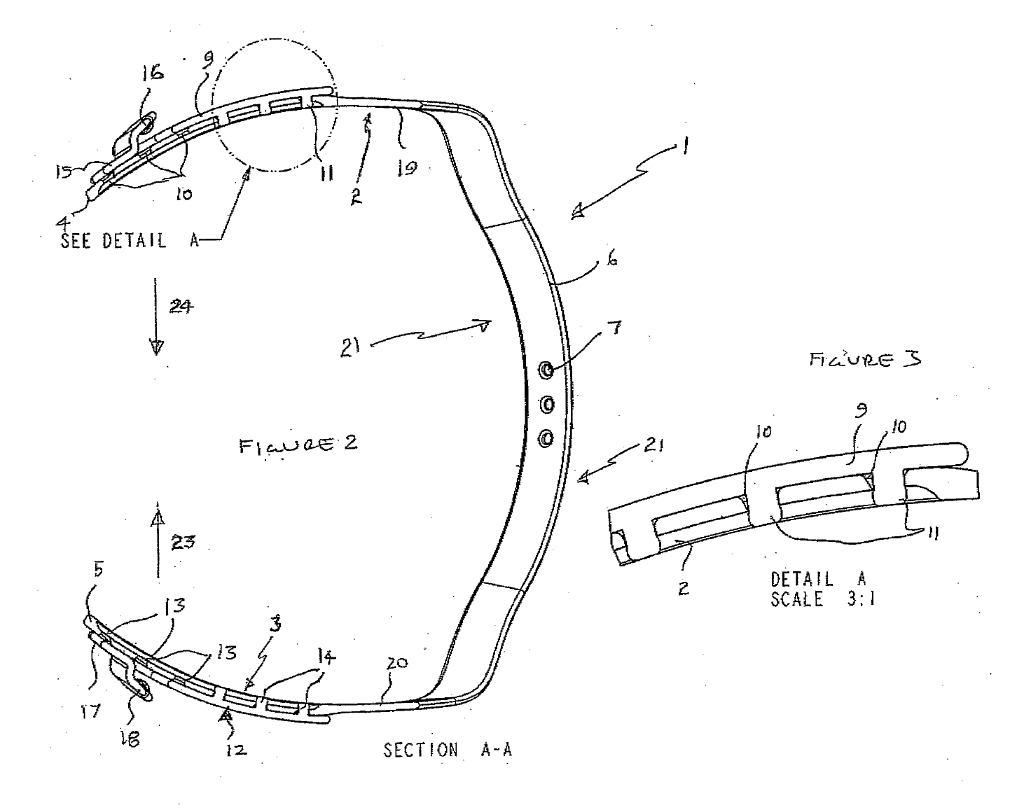

FIG. 1 shows a perspective view of a mask mounting headgear harness according to one embodiment. Harness 1 comprises al body 21 including side arm members 2 and 3 each terminating in respective free ends 4 and 5. Body 21 further comprises a region 6 Which allows selective adjustment of the overall length of the body 21 to allow the harness 1 to accommodate different head sizes of a wearer. At region 6 which locates the back of the head, there are provided a series of eyelets 7 which receive studs 8 in a female male engagement, to allow said selective adjustment of length. Arm member 2 includes an extension member 9 which is detachably fixed thereto to enable selective adjustment of length of arm 2. Extension member 9 is provided with a series of eyelets 10 which receive studs 11 (obscured see FIG. 2) allowing a second area for length adjustment. Arm member 3 includes an extension member 12 which is detachably fixed to enable selective adjustment of length of arm 3. Arm 3 is provided with a series of eyelets 13 which receive studs 14 (obscured) allowing a third longitudinal length adjustment. Extension member 9 includes a connection assembly 15 including retaining tab 16 which receives and retains a connector (not shown) which enables attachment of a respirator mask to harness 1. Extension member 12 includes a connection assembly 17 including retaining tab 18 which receives and retains a connector (not shown) which enables attachment of a respirator mask to harness 1. Region 6 of, harness 1 fits behind the head of a wearer. Arm 2 locates along one side a wearer's head and arm 3 locates along the opposite side of the wearer's head. Arm 2 includes a contoured portion 19 which is shaped to skirt above a right ear of a wearer. Likewise arm 3 includes a contoured portion 20 which is also shaped to skirt above a left ear of a wearer. Support for harness 1 may be provided by resting on a wearers ear, by a sufficient clamping force on sides of the head of the wearer by arms 2 and 3 creating a gripping force on the face of a wearer or by a combination of those two regimes. The stiffer the harness material the greater the available clamping force.

FIG. 2 shows with corresponding numbering, a top view of the harness 1 of FIG. 1. From the top view it can be seen that arm member 2 includes an extension member 9 which is detachably fixed thereto to enable selective adjustment of length of arm 2. Extension member 9 is provided with a series of eyelets 10 which receive studs 11 allowing a length adjustment. Arm member 3 includes an extension member 12 which is detachably fixed to enable selective adjustment of length of arm 3. Arm 3 is provided with a series of eyelets 13 which receive studs 14 allowing a longitudinal length adjustment. Extension members 9 and 12 made be peeled away from respective arms 2 and 3 and reset by respective engagements between eyelets 10 and studs 11 and eyelets 13 and studs 14.

Retaining tab 16 receives and retains a connector (not shown) which enables attachment of a respirator mask to harness 1 and retaining tab 18 receives and retains a connector (not shown) which enables attachment of a respirator mask to harness 1. Arm 2 locates along one side a wearer's head and arm 3 locates along the opposite side of the wearer's head and both due to their elasticity provide a gripping or clamping force on the head of a wearer. The preferred gripping force is applied in the direction of opposing arrows 23 and 24.

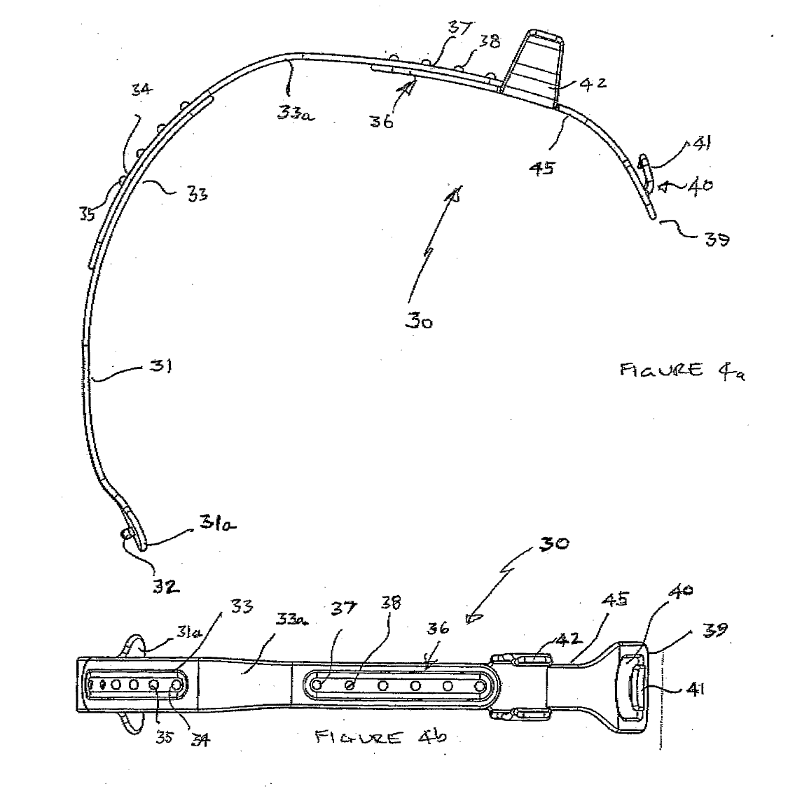

FIG. 3 shows with corresponding numbering, an enlarged view of the connection between the headgear side arm 2 and extension member 9.

FIG. 4 shows a side elevation of an overhead connecting arm assembly 30 which is an optional attachment to headgear 1. Arm assembly 30 includes a connection arm 31 terminating in free end 31a which includes studs 32 which engage, a corresponding eyelet 7 (see FIG. 1) in headgear body 21 to enable connection thereto. Arm 31 includes at opposite end 33 a series of studs 35 which selectively engage eyelets 34 in connection arm 33a. Studs 35 can be selectively located in eyelets 34 to enable length adjustment of assembly 30 by altering the relative positions of arms 33 and 33a. Arm assembly 30 further comprises a second extension connection 36 which engages arm 33a with arm 45. Arm. 33a includes a series of studs 38 which selectively penetrate corresponding eyelets 37. Studs 38 can be selectively located in eyelets 37 to enable a second length adjustment of arm 30 by relative adjustments between arm 33a and arm 45. Arm assembly 30 terminates at free end 39 in connection assembly 40 which includes tab 41 which receives a connector extending from a face mask to be attached to harness 1. Arm assembly 30 further comprises saddle 42 which can accommodate an air tube which is in communication with a respirator mask(not shown).

FIG. 4b shows with corresponding numbering, a top view of the overhead connecting arm assembly 30. As can be seen, arm 45 can be extended relative to arm 33a by selected engagement of

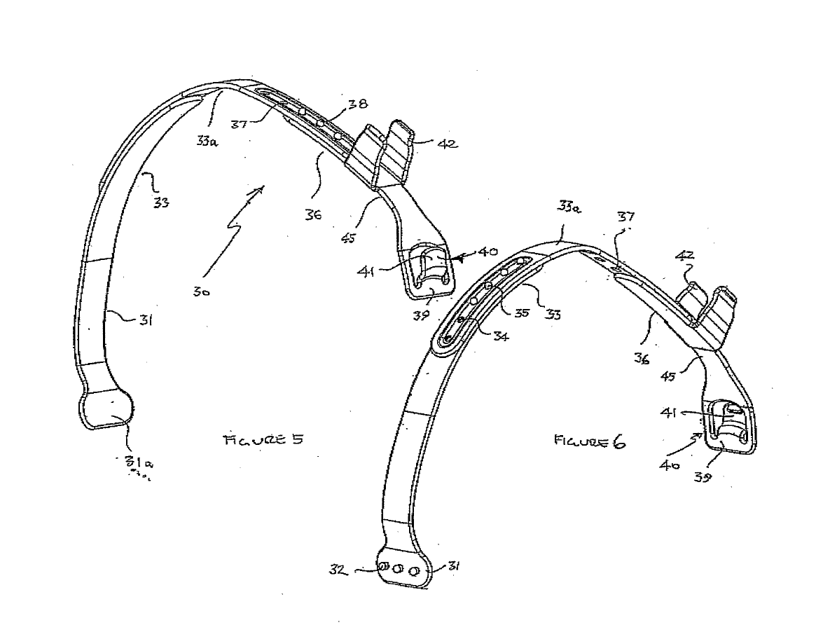

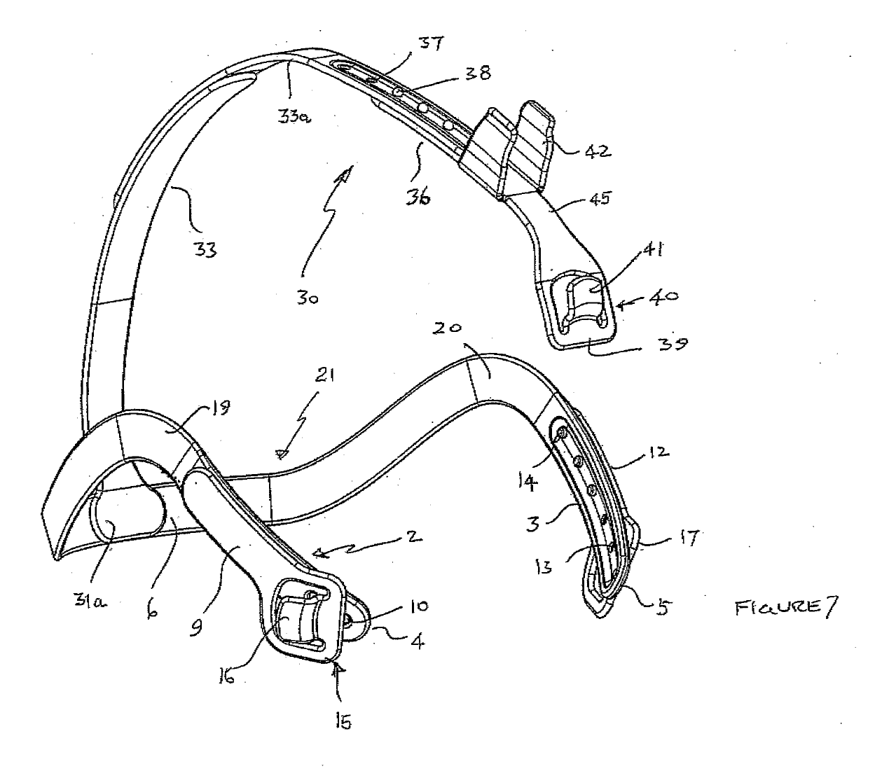

FIG. 5 shows with corresponding numbering a front perspective view of the overhead connecting arm 30 of FIG. 4. FIG. 6 shows with corresponding numbering a rear perspective view of the connecting arm 30 of FIG. 5.

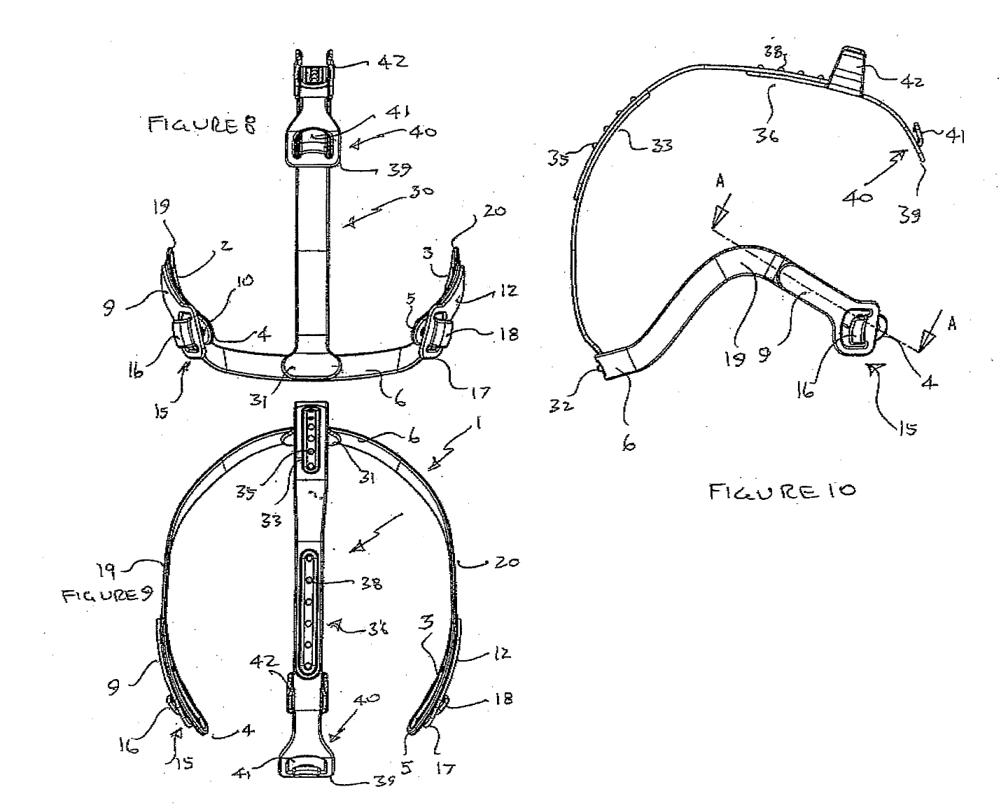

FIG. 7 shows with corresponding numbering a front perspective view of the harness 1 of FIG. 1 with the overhead connecting arm 30 of FIGS. 4-6 connected thereto. FIG. 8 shows a top view of the harness 1 of FIG. 7.

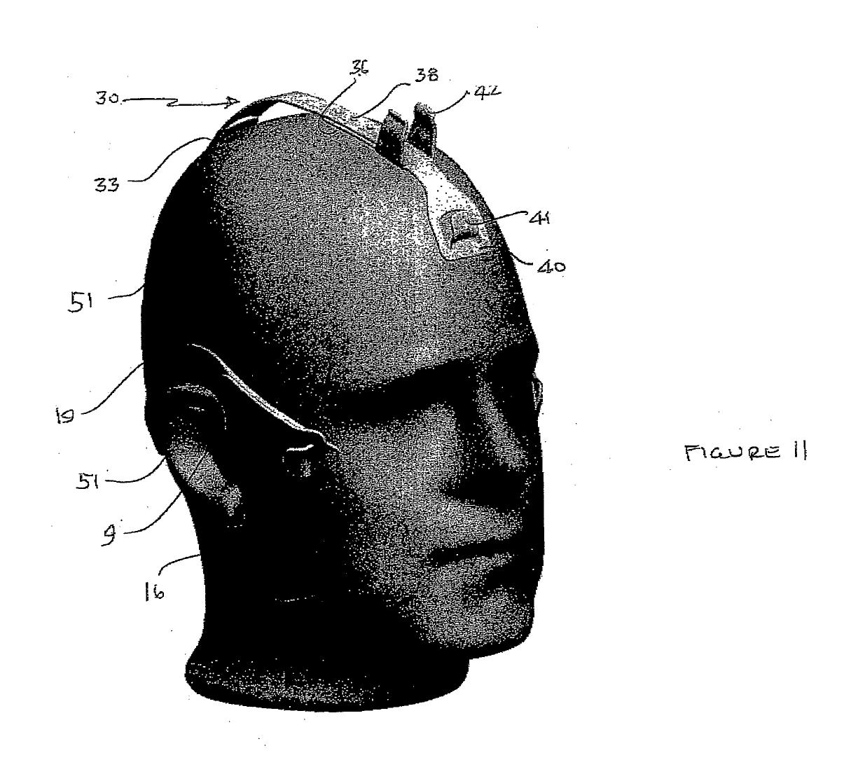

FIG. 9 shows a side view of the harness 1 of FIG. 7.

FIG. 10 shows with corresponding numbering for corresponding parts, the harness of FIG. 7 mounted on the head 50 of a wearer. It can be seen that with reference to contoured portion 19 that it travels above ear 51, preferably avoiding the ear altogether. Contoured portion 19 is shown skirting above the top of the wearer's ear 51. Alternatively, the ear can contribute to support of harness 1. Arm 30 passes over the top region 52 of head 50 to provided additional locating support for harness 1. Connection assembly 40 of arm 30, and respective connection assemblies 15 and 17 of arms 2 and 3 receive connectors from a mask (not shown) so that three point support is provided.

Forces applied to the mask from the harness under the tension of the mask connectors, are distributed around the body of the flexible mask using a mask shape forming component, which is integral to (or may be attached to) the sidewall of the mask. The shape-forming components (webs) are designed to distribute distortional forces to a substantial portion of the mask sidewall. These forces are then transmitted from the mask sidewall to the remainder of the mask body.

Since harness 1 has a number of extension members, the extent of compressive force applied to the face of a wearer can be adjusted by length adjustments in the harness members which in turn will result in a compressive force on the face of a wearer. It is possible to adjust the relative size of those forces at different points around the face contacting portion of the mask by altering the extension lengths of the free end extension members of the harness. The shorter the length of the harness members the larger will be the mask compression forces.

The patient can sleep with the mask and harness in contact with objects such as a pillow. In comparison with conventional mask harnesses, the mask can be conveniently unhooked form the harness with minimal or no disturbance to the patient as the harness is self supporting and rigid enough to retain its position upon removal of the mask. This is particularly advantageous in the case of children as the harness can be set to a child's head and the mask removed as required. Although partially rigid harnesses have been used in the past, they are unable to stay self supporting on the head. With conventional harnesses the tensioning adjustments are performed in conjunction with the mask mounting. According to the harness of the present invention the harness can be adjusted and set For a particular wearer and the mask fitted to the pre set harness. Materials which may be used for the harness include plastics, metals of a combination of the two.

It will be appreciated by persons skilled in the art that numerous variations and/or modifications may be made to the invention as shown in the specific embodiments without departing from the spirit or scope of the invention as broadly described. The present embodiments are therefore, to be considered, in all respects as illustrative and not restrictive.

1-35. (canceled)

36. A self supporting head mounted harness for receiving and retaining patient care equipment or devices, the harness comprising:
a harness body having at least two face engaging members for respectively engaging opposing sides of the face of a wearer;
the two face engaging members each terminating at a free end, coupled to at least one connector adapted to receive and retain the equipment or devices;
at least one length adjustable assembly coupled to a free end, for providing length adjustment to accommodate different sized wearers' heads, the length adjustable assembly comprising a detachable extension arm which is length adjustable relative to the free end of the face engaging members; and, wherein, the face engaging members of the harness body are formed from a material which is capable of exerting at least a slight gripping or clamping force against the head of the wearer such that the harness remains self supporting on the wearer's head independent of engagement with or disengagement from the equipment or devices.

37. A harness according to claim 36, wherein the face engaging members are capable of elastic displacement to apply a clamping force biased to the head of the user to hold the harness body in place on the head.

38. A harness according to claim 36, further comprising two extension arms which are adjustable to accommodate the shape and size of a wearer's head.

39. A harness according to claim 36, further comprising a third overhead member which terminates in a free end connection assembly.

40. A harness according to claim 39, wherein the overhead member comprises an adjustable arm which enables adjustments in the length of the overhead member to accommodate different patient head sizes.

41. A harness according to claim 36, wherein a device attached to the harness is a respirator mask which locates on the face of a user.

42. A harness according to claim 41, wherein the connections between the adjustable arms and the mask are such that loadings applied to the mask via the arms are distributed evenly so that distortions of the mask under load do not compromise a required airtight seal between the user's face and the face contacting part of the mask.

43. A harness according to claim 41, wherein the harness is coupled to a pipe on a manifold of the respirator mask.

44. A harness according to claim 41, wherein the overhead member engages a web or nasal arch strap which is integral to a portion of a gas delivery pipe associated with the mask.

45. A harness according to claim 44, wherein the manifold is sufficiently flexible to collapse towards the patient's nose when a moderate external force is applied thereto.

46. A harness according to claim 45 wherein, the mask includes straps or webs having openings for engaging the connectors of the free ends of the face engaging members of the harness.

47. A harness according to claim 46, wherein the openings are eyelets or slots located at respective free ends of the webs.

48. A harness according to claim 47, wherein the webs are integral with the side walls of the mask.

49. A harness according to claim 48, wherein the webs extend away from side walls of the mask, with at least one strap on each side of the mask and one strap running along the nasal arch of the user's forehead.

50. A harness according to claim 41, wherein the mask is retained to the harness by a preset tensile force in the webs to induce a required degree of mask compression on the face of a wearer.

51. A harness according to claim 41, wherein the harness comprises a plurality of adjustable attachment points which can be adjusted in length so as to change distribution of forces to various areas of the mask.

52. A harness according to claim 51, wherein the mask manifold is flexible enough to collapse toward the patients nose when a moderate external force is applied to it.

53. A harness according to claim 36, wherein the side members of the harness which engage the user's face, follow the general shape of the contours of a patient's head and cheeks.

54. A harness according to claim 36, wherein the side members comprise a region which is deployed around the wearer's ears.

55. A harness according to claim 36, wherein the device attached to the harness is an air pipe.

56. A harness according to claim 36, wherein the device attached to the harness is a monitor for measuring a patient health parameter.

57. A harness according to claim 36, wherein the device attached to the harness is a sensor for determining and recording a patient health parameter.

58. A harness according to claim 36, wherein the device attached to the harness is a nasal prong or nasal pillow.

59. A harness according to claim 36, wherein, the device is a tube delivering a gas such as oxygen to the patient.

60. A harness assembly for a respirator mask mounted on a user's face for supplying gas under pressure to an airway of a human, the mask comprising having a flexible manifold shell, and being made of a flexible material, the manifold being adapted for connection to a gas delivery pipe, the assembly comprising:
at least two side walls which are at least partially comprised of portions of the manifold shell;
a first mask shape forming element for distributing distortional forces to a substantial portion of a first side wall that attaches to, or is integral with, a significant portion of that one side wall of the mask; and
a second mask shape forming element for distributing distortional forces to a substantial portion of a second side wall that attaches to, or is integral with, a significant portion of the second side wall of the mask, each mask shape forming element being coupled to, or being coupleable to, a strap;
a flexible face contacting element defining an orifice to accommodate the nose of the human;
the harness assembly further comprises:
a body having at least two members which each terminate at a free end; the free ends having connectors adapted to receive and retain a part of the mask; wherein the harness body is formed from a material which is capable of exerting at least a slight gripping or clamping force against the head of a wearer such that when the mask is retained by the harness or removed from the harness the harness is self supporting on the wearer's head.

61. A harness assembly according to claim 60, wherein the connectors receive and retain a part of the mask.

62. A harness assembly according to claim 60, further comprising an overhead attachment member for detachable engagement with the head mounted harness for mounting a respirator mask on the face of the user; wherein the harness body has a material stiffness sufficient to enable the harness to be retained and self supported on the head of a user independent of whether or not the mask is attached to the harness; the overhead attachment member including an adjustable arm which enables adjustments in the length of the overhead member to accommodate different patient head sizes.
